(19) United States
(12) Patent Application Publication
(10) Pub. No.: US 2018/0117036 A1
(43) Pub. Date: May 3, 2018

(54) ORAL PHARMACEUTICAL COMPOSITION FOR INCREASING HYPOXIA TOLERANCE

(71) Applicant: CHANGZHOU HI-TECH DISTRICT MULTIPLE DIMENSION INDU, Jiangsu (CN)

(72) Inventors: Hebing XIE, Jiangsu (CN); Qingyi LI, Jiangsu (CN); Shuhua GU, Jiangsu (CN); Weihong LV, Jiangsu (CN)

(21) Appl. No.: 15/857,494

(22) Filed: Dec. 28, 2017

Related U.S. Application Data
(63) Continuation of application No. 14/889,613, filed on Nov. 6, 2015.

(30) Foreign Application Priority Data
May 6, 2013 (CN) 201310161769.7

(51) Int. Cl. A61K 31/495; A61K 9/00; A61K 31/205

(52) U.S. Cl. CPC A61K 31/495; A61K 9/0053; A61K 2300/00; A61K 31/205

(57) ABSTRACT

The present invention relates to an oral pharmaceutical composition for increasing hypoxia tolerance, characterized in that the pharmaceutical composition comprises active ingredient L-carnitine or derivative thereof or pharmaceutically acceptable salt thereof, active ingredient trimetazidine or pharmaceutically acceptable salt thereof, and a pharmaceutically acceptable auxiliary material, and 100:1 is the weight ratio of L-carnitine or derivative thereof or pharmaceutically acceptable salt thereof and trimetazidine or pharmaceutically acceptable salt thereof.

### CROSS-REFERENCE TO RELATED APPLICATIONS

This application is a continuation of U.S. Ser. No. 14/889,613, filed on Nov. 6, 2015, having the same title, and having the same inventors, and which is incorporated herein in by reference in its entirety; which application is a 371 PCT national application claiming priority to PCT/CN2014/075896, filed Apr. 22, 2014, having the same title, and having the same inventors, and which is incorporated herein in by reference in its entirety; which application claims the benefit of priority from Chinese patent application number 201310161769.7, filed May 6, 2013, having the same title, and having the same inventors, now pending, and which is incorporated herein by reference in its entirety.

### TECHNICAL FIELD

The present invention relates to pharmaceutical formulations and in particular relates to an oral pharmaceutical composition for increasing hypoxia tolerance.

### BACKGROUND ART

Hypoxia refers to a pathological process in which abnormal changes in metabolism, functions and morphological structures of a tissue occur due to inadequate oxygen supply or dysfunction in oxygen use. Hypoxia consists of 4 types, namely hypotonic hypoxia, hemic hypoxia, circulatory hypoxia and histogenous hypoxia, in which hemic hypoxia and histogenous hypoxia are dysoxidative hypoxia while hypotonic hypoxia and circulatory hypoxia are caused by inadequate oxygen supply.

Hypoxia generates a lot of free radical which damage stability of mitochondrial membrane, hurt body tissues functions and structures, and cause energy metabolism dysfunctions, with clinical manifestation as normal hypoxia manifestation including, among others, dizziness, encephalalgia, tinnitus, dim sight, limb asthenia, lower exercise performance, thought slowness, memory deterioration, nausea, vomit, palpitation, brachypnea, tachypnea and fast but weak heart beat, or as serious diseases including, among other, myocardial infarction, angina pectoris, pneumonedema, encephaledema, shock, respiratory failure, cerebral apoplexy, optic nerve injury and cranial nerves injuries.

Medicines for increasing hypoxia tolerance which is mostly used in clinic are diuretics such as acetazolamide, and adrenocortical hormone agents such as dexamethasone and aminophylline. However, these medicines are not suitable for long-term administration due to their toxic side effect. For example, long term administration of acetazolamide tends to cause adverse effect such as body electrolyte disorder. In addition, traditional Chinese medicine (TCM) preparations comprising Rhodiola rosea are usually used in hypoxia prophylaxis and treatment. These TCM sustained release formulations facilitate enhancement of body adaptability to hypoxia and reduction of stress response, so as to increase hypoxia tolerance. However, these TCMs take effect slowly and provide limited effect. Chinese patent under application number 200310104871.X disclosed that L-carnitine presents effective prophylaxis and treatment of altitude sickness. However, there has been no report on its clinical application so far.

Apparently, a medicine that is suitable for long term administration, combination of prophylaxis and treatment, and effective increase of hypoxia tolerance, without presenting obvious adverse effect, is still in need.

### SUMMARY OF INVENTION

One objective of the present invention is to provide an oral pharmaceutical composition which is clinically convenient, orally administrable and capable of effectively increasing hypoxia tolerance, the pharmaceutical composition comprising active ingredient L-carnitine or derivative thereof or pharmaceutically acceptable salt thereof, active ingredient trimetazidine or pharmaceutically acceptable salt thereof, and a pharmaceutically acceptable auxiliary material, and 100:1 is the weight ratio of L-carnitine or derivative thereof or pharmaceutically acceptable salt thereof and trimetazidine or pharmaceutically acceptable salt thereof.

The second objective of the present invention is to provide use of the oral pharmaceutical composition in preparation of medicines for increasing hypoxia tolerance.

The third objective of the present invention is to provide use of the oral pharmaceutical composition in preparation of medicines for increasing blood oxygen saturation.

During extensive animal experiments, researchers of the present invention unexpectedly found that trimetazidine or pharmaceutically acceptable salt thereof and L-carnitine or derivative thereof or pharmaceutically acceptable salt thereof can be combined in a predetermined proportion in administration or into a composition, which can increase blood oxygen saturation of hypoxic rats and extend the survival period of mice in hypoxic condition.

Researchers of the present invention prepared oral pharmaceutical formulations, such as oral tablets, granules and oral liquid, with trimetazidine or pharmaceutically acceptable salt thereof, L-carnitine or derivative thereof or pharmaceutically acceptable salt thereof and a specific pharmaceutically acceptable auxiliary material in a predetermined weight proportion.

Hypoxia refers to a pathological condition in which abnormal changes in metabolism, functions and morphological structures of a tissue occur due to inadequate oxygen supply or dysfunction in oxygen use. In the present invention, hypoxia particularly refers to a pathological condition in which abnormal changes in metabolism, functions and morphological structures of a tissue occur due to inadequate oxygen supply.

In the present invention, clinical manifestation of hypoxia includes normal hypoxia manifestation including, among others, dizziness, encephalalgia, tinnitus, dim sight, limb asthenia, lower exercise performance, thought slowness, memory deterioration, nausea, vomit, palpitation, brachypnea, tachypnea and fast but weak heart beat, and serious diseases including, among other, myocardial infarction, angina pectoris, pneumonedema, encephaledema, cerebral apoplexy, shock, respiratory failure, optic nerve injury and cranial nerves injuries.

In the present invention, increasing hypoxia tolerance refers to prophylaxis and treatment of symptoms and diseases with clinical manifestation of hypoxia, and in particular refers to prophylaxis and treatment of normal hypoxia manifestation including, among others, dizziness, encephalalgia, tinnitus, dim sight, limb asthenia, lower exercise performance, thought slowness, memory deterioration, nausea, vomit, palpitation, brachypnea, tachypnea and fast but weak heart beat.

The present invention provides an oral pharmaceutical composition for increasing hypoxia tolerance, the pharmaceutical composition comprising active ingredient L-carnitine or derivative thereof or pharmaceutically acceptable salt thereof, active ingredient trimetazidine or pharmaceutically acceptable salt thereof, and a pharmaceutically acceptable auxiliary material, and 100:1 is the weight ratio of L-carnitine or derivative thereof or pharmaceutically acceptable salt thereof and trimetazidine or pharmaceutically acceptable salt thereof.

In the oral pharmaceutical composition according to the present invention, the L-carnitine or derivative thereof or pharmaceutically acceptable salt thereof is selected from L-carnitine, acetyl-L-carnitine, propionyl-L-carnitine and pharmaceutically acceptable salts thereof, and is preferably L-Carnitine; the pharmaceutically acceptable salts of trimetazidine, L-carnitine or derivatives thereof comprise their salts formed with hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid and p-toluene sulfonic acid.

A particularly preferred example of the oral pharmaceutical composition according to the present invention is a tablet which comprises active ingredient L-carnitine or derivative thereof or pharmaceutically acceptable salt thereof, active ingredient trimetazidine or pharmaceutically acceptable salt thereof, and a pharmaceutically acceptable auxiliary material, and 100:1 is the weight ratio of L-carnitine or derivative thereof or pharmaceutically acceptable salt thereof and trimetazidine or pharmaceutically acceptable salt thereof.

A particularly preferred example of the oral pharmaceutical composition according to the present invention is a granule which comprises active ingredient L-carnitine or derivative thereof or pharmaceutically acceptable salt thereof, active ingredient trimetazidine or pharmaceutically acceptable salt thereof, and a pharmaceutically acceptable auxiliary material, and 100:1 is the weight ratio of L-carnitine or derivative thereof or pharmaceutically acceptable salt thereof and trimetazidine or pharmaceutically acceptable salt thereof.

A particularly preferred example of the oral pharmaceutical composition according to the present invention is an oral liquid which comprises active ingredient L-carnitine or derivative thereof or pharmaceutically acceptable salt thereof, active ingredient trimetazidine or pharmaceutically acceptable salt thereof, and a pharmaceutically acceptable auxiliary material, and 100:1 is the weight ratio of L-carnitine or derivative thereof or pharmaceutically acceptable salt thereof and trimetazidine or pharmaceutically acceptable salt thereof.

The oral pharmaceutical composition according to the present invention is a formulation for oral administration, including granules, tablets, capsules, oral liquid, preferably tablets, granules and oral liquid. The oral pharmaceutical composition can also use combined package.

The present invention further provides use of a composition comprising L-carnitine or derivative thereof or pharmaceutically acceptable salt thereof, trimetazidine or pharmaceutically acceptable salt thereof, and a pharmaceutically acceptable auxiliary material, in preparation of a medicine for increasing hypoxia tolerance. In the medicine, the ratio of L-carnitine or derivative thereof or pharmaceutically acceptable salt thereof and trimetazidine or pharmaceutically acceptable salt thereof is 66-4000:1, preferably 66-100:1, more preferably 100:1. Daily dosage for an adult is as follows: 10-500 mg/kg of L-carnitine or derivative thereof or pharmaceutically acceptable salt thereof and 0.1-1 mg/kg of trimetazidine or pharmaceutically acceptable salt thereof. The L-carnitine or derivative thereof or pharmaceutically acceptable salt thereof is selected from L-carnitine, acetyl-L-carnitine, propionyl-L-carnitine and pharmaceutically acceptable salts thereof. The pharmaceutically acceptable salts of trimetazidine, L-carnitine or derivatives thereof comprise salts formed with hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid and p-toluene sulfonic acid.

The present invention further provides use of a composition comprising L-carnitine or derivative thereof or pharmaceutically acceptable salt thereof, trimetazidine or pharmaceutically acceptable salt thereof, and a pharmaceutically acceptable auxiliary material, in preparation of a medicine for increasing blood oxygen saturation. In the medicine, the ratio of L-carnitine or derivative thereof or pharmaceutically acceptable salt thereof and trimetazidine or pharmaceutically acceptable salt thereof is 50-300:1, preferably 100:1. Daily dosage for an adult is as follows: 10-500 mg/kg of L-carnitine or derivative thereof or pharmaceutically acceptable salt thereof and 0.1-1 mg/kg of trimetazidine or pharmaceutically acceptable salt thereof. The L-carnitine or derivative thereof or pharmaceutically acceptable salt thereof is selected from L-carnitine, acetyl-L-carnitine, propionyl-L-carnitine and pharmaceutically acceptable salts thereof. The pharmaceutically acceptable salts of trimetazidine, L-carnitine or derivatives thereof comprise salts formed with hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid and p-toluene sulfonic acid.

### EMBODIMENT

The following examples are provided for further explaining the present invention only and do not intend to limit the scope of the invention.

### Example 1

Observation of influence of oral administration of different dosage combination of L-carnitine+trimetazidine to hypoxic mice in normal pressure trimetazidine hydrochloride: 0.15, 0.75, 1.5, 3, 6 and 9 mg/kg, equivalent to human daily dosage of 1, 5, 10, 20, 40 and 60 mg;

L-carnitine: 600 mg/kg, equivalent to human daily dosage of 4 g; 70 male mice are selected, each of a weight of 20±2 g. The mice are divided into 10 groups randomly based on weight, 10 for each group, and are given oral administration at a dosage of 20 ml/kg, while the control group is given isovolumetric normal saline, both once a day for consecutive 7 days. In one hour after the final administration, each group of mice are placed in wide mouth bottles of a volume of 160 ml, into which 5 g of soda lime has been pre-added. One bottle contains one mouse and its cap is sealed with Vaseline. Taking death of mice as index, putting down the survival time of mice and taking a 20% or more extension of survival time as significant effect. Please refer to table 1 for the results.

TABLE 1
Comparison of Survival Time under Normal Pressure in Hypoxic
Condition (n = 10, x̅ ± S)

| | Survival | Extended Survival |
|---|---|---|
| Group/Dosage (mg/kg) | Time (min) | Time (%) |
| Control Group | 21.5 ± 5.6 | — |
| L-carnitine 600 + | 29.0 ± 2.1 | 34.9 |
| trimetazidine hydrochloride 0.15 | | |
| L-carnitine 600 + | 29.5 ± 2.9 | 37.2 |
| trimetazidine hydrochloride 0.75 | | |
| L-carnitine 600 + | 30.4 ± 3.3** | 41.4 |
| trimetazidine hydrochloride 1.5 | | |
| L-carnitine 600 + | 31.8 ± 1.4* | 47.9 |
| trimetazidine hydrochloride 3 | | |
| L-carnitine 600 + | 35.4 ± 4.5** | 64.7 |
| trimetazidine hydrochloride 6 | | |
| L-carnitine 600 + | 35.0 ± 5.7** | 62.8 |
| trimetazidine hydrochloride 9 | | |

Remarks: as compared to the control group,
*P < 0.05,
**P < 0.01.

The results show that: the composition of L-carnitine and trimetazidine hydrochloride (66-40000:1) can extend the survival time of mice in hypoxia and lower weight ratio presents more significant effect. The most significant effect is achieved when the weight ratio of L-carnitine and trimetazidine hydrochloride is 100:1.

### Example 2

Comparison of L-carnitine 600 mg/kg+trimetazidine hydrochloride 6 mg/kg and separate intragastric administration on mice in hypoxia under atmospheric pressure 40 male mice are selected, each of a weight of 20±2 g. The mice are divided into 4 groups randomly based on weight, 10 for each group, and are given intragastric administration at a dosage of 20 ml/kg, while the control group is given isovolumetric normal saline, both once a day for consecutive 7 days. In one hour after the final administration, each group of mice are placed in wide mouth bottles of a volume of 160 ml, into which 5 g of soda lime has been pre-added. One bottle contains one mouse and its cap is sealed with Vaseline. Taking death of mice as index and putting down the survival time of mice. Please refer to table 2 for the results.

TABLE 2
Comparison of Survival Time under Normal Pressure in Hypoxic
Condition (n = 10, x̅ ± S)

| | Survival | Extended Survival |
|---|---|---|
| Group/Dosage (mg/kg) | Time (min) | Time (%) |
| Control Group | 21.5 ± 5.6 | — |
| trimetazidine hydrochloride 6 | 26.0 ± 5.2* | 20.9 |
| L-carnitine 600 | 28.6 ± 4.8* | 33.0 |
| trimetazidine hydrochloride | 36.0 ± 7.1** | 67.5 |
| 6 + L-carnitine 600 | | |

Remarks: as compared to the control group,
*P < 0.05,
**P < 0.01.

The results show that: as compared to separate use of L-carnitine or trimetazidine hydrochloride, the composition significantly extends the survival time of mice (P<0.01) Combination of the two medicines provides synergistic effect. Therefore, it shows that compound preparation is better than single preparation.

### Example 3

Observation of influence of administration of different dosage combination of L-carnitine+trimetazidine to hypoxic rats in normal pressure trimetazidine hydrochloride: 2, 4 and 6 mg/kg, equivalent to human daily dosage of about 20, 40 and 60 mg; L-carnitine: 200, 400 and 600 mg/kg, equivalent to human daily dosage of about 2, 4 and 6 g; 70 Wister rats are selected, each of a weight of 150 g˜190 g. The rats are divided into 7 groups randomly: normoxic control group: raised and collected in plain area; acute hypoxia group: animals are placed in a low pressure oxygen cabin having a cabin oxygen partial pressure of 11.01 Kpa (equivalent to about oxygen partial pressure at 5000 m above sea level). In decompression hypoxia for 3 days, the animals are further placed in a low pressure oxygen cabin having a cabin oxygen partial pressure of 13.25 Kpa (equivalent to about oxygen partial pressure at 4000 m above sea level) for sampling [Yue ZHENG, Yang J I, Animal Models Commonly Used in Researches for Increasing Hypoxia Tolerance and Medicines for Increasing Hypoxia Tolerance, Pharm J Chin PLA, 2010, 26(2):170-173]; Administration Group: intragastric administration at a dosage of 20 ml/kg for seven days since four days before entering the low pressure oxygen cabin. Collecting data and samples in a low pressure oxygen cabin having a cabin oxygen partial pressure at 13.25 Kpa (equivalent to about oxygen partial pressure at 4000 m above sea level). All animals are freely eating and drinking.

Hemodynamic measurement: at corresponding time point, cardiac catheters are inserted to pulmonary artery via right external jugular vein and to aorta and left ventricle via left common carotid artery of each group of animals; a four-channel physiology recorder is used to record heart rate (HR), pulmonary artery pressure (PAP), systolic aortic pressure (SAP), diastolic aortic pressure (DAP), left ventricle systolic pressure (LVSP), left ventricle diastolic pressure (LVEDP), and the maximum increase rate of left ventricle pressure (+dp/dtₘₐₓ). Please refer to table 3 for the results.

TABLE 3
Influence of different dosage combination of L-carnitine and trimetazidine to
haematological index of rats in simulated plateau hypoxia condition (n = 10, x̅ ± S)

| oup/Dosage | PAP | | DAP | LVSP | +dp/dtₘₐₓ | HR |
|---|---|---|---|---|---|---|
| (mg/kg) | (Kpa) | SAP (Kpa) | (Kpa) | (Kpa) | (KPa) | (heat/min) |
| Normoxic | 3.5 ± 0.6 | 15.8 ± 1.6 | 10.5 ± 2.8 | 16.9 ± 1.6 | 664 ± 83 | 360 ± 40 |
| Control | | | | | | |
| Group | | | | | | |
| Acute | 5.3 ± 0.7 | 22.9 ± 3.7 | 15.6 ± 3.2 | 25.5 ± 3.0 | 695 ± 72 | 377 ± 50 |
| Hypoxia | | | | | | |
| Group | | | | | | |
| L-carnitine | 4.7 ± 0.8* | 19.7 ± 2.3* | 13.6 ± 3.5* | 21.9 ± 2.8* | 623 ± 77* | 375 ± 52 |
| 200 + | | | | | | |
| trimetazidine | | | | | | |
| hydrochloride 4 | | | | | | |
| L-carnitine | 4.2 ± 0.7** | 18.2 ± 2.5** | 12.5 ± 2.6** | 20.1 ± 2.5** | 561 ± 43** | 370 ± 45 |
| 400 + | | | | | | |
| trimetazidine | | | | | | |
| hydrochloride 4 | | | | | | |
| L-carnitine | 3.9 ± 0.5** | 17.5 ± 1.9** | 11.2 ± 2.4** | 18.5 ± 1.8** | 517 ± 60** | 368 ± 48 |
| 600 + | | | | | | |
| trimetazidine | | | | | | |
| hydrochloride 4 | | | | | | |
| L-carnitine | 3.6 ± 0.5** | 16.1 ± 3.4** | 10.8 ± 2.1** | 17.2 ± 1.9** | 494 ± 54** | 365 ± 50 |
| 600 + | | | | | | |
| trimetazidine | | | | | | |
| hydrochloride 6 | | | | | | |
| L-carnitine | 5.1 ± 0.6* | 20.9 ± 3.5* | 14.3 ± 3.1* | 23.2 ± 3.2* | 657 ± 75* | 375 ± 58 |
| 600 + | | | | | | |
| trimetazidine | | | | | | |
| hydrochloride 2 | | | | | | |

Remarks: as compared to the acute hypoxia group, *P < 0.05, **P < 0.01.

Blood gas analysis: collecting 1 ml of blood from aorta; heparin anticoagulation; measuring blood gas index including, among others, blood oxygen partial pressure PaO₂ and oxygen saturation SaO₂. Please refer to table 4 for the results.

TABLE 4
Influence of different dosage combination of L-carnitine
and trimetazidine to blood gas analysis of rats in simulated
plateau hypoxia condition (n = 10, x̅ ± S)

| Group/Dosage (mg/kg) | PaO₂ (Kpa) | SaO₂ (%) |
|---|---|---|
| Normoxic Control Group | 12.2 ± 2.4 | 91.4 ± 6.3 |
| Acute Hypoxia Group | 5.5 ± 1.5 | 63.7 ± 13.8 |
| L-carnitine 200 + | 6.9 ± 1.9** | 72.5 ± 13.2** |
| trimetazidine hydrochloride 4 | | |
| L-carnitine 400 + | 8.2 ± 2.0** | 83.6 ± 10.1** |
| trimetazidine hydrochloride 4 | | |
| L-carnitine 600 + | 8.1 ± 1.5** | 78.9 ± 16.5** |
| trimetazidine hydrochloride 4 | | |
| L-carnitine 600 + | 9.8 ± 2.5** | 90.7 ± 14.8** |
| trimetazidine hydrochloride 6 | | |
| L-carnitine 600 + | 6.3 ± 1.5** | 70.4 ± 11.6** |
| trimetazidine hydrochloride 2 | | |

Remarks: as compared to the acute hypoxia group,
**P < 0.01.

The results show that:

According to Table 3, all the Administration Groups can significantly increase hemodynamic indexes, which shows that it has the effect of increasing hypoxia tolerance, and the effect of the combination of L-carnitine 600 mg/kg and trimetazidine hydrochloride 6 mg/kg is the closest to that of the normoxic control group.

According to Table 4, each administration group can significantly increase arterial blood oxygen partial pressure and oxygen saturation of hypoxic rats (P<0.01), which shows that the composition according to the present invention can increase bonding strength of hemoglobin and oxygen, oxygen carrying capacity and hypoxia tolerance. The effect of the combination of L-carnitine 600 mg/kg and trimetazidine hydrochloride 6 mg/kg is the closest to that of the normoxic control group.

### Example 4: Study of Selection of Auxiliary Material of Normal Tablets

Based on physiochemical properties of L-carnitine and characteristics of the dosage form, namely L-carnitine is a flaky crystal and extremely easy to absorb moisture, a sustained release auxiliary material that has moisture absorption resistance shall be adopted. The inventors selected auxiliary materials including, among other, microcrystalline cellulose, calcium carbonate, cross-linked polyvinylpyrrolidone and talcum powder via a great amount of Pharmaceutics Experiments, among which microcrystalline cellulose and calcium carbonate are excipients, cross-linked polyvinylpyrrolidone is a disintegrant, and talcum powder can be used as framework material to increase formability of granules and tablets and as lubricant to avoid sticking and picking during the process of tableting.

Microcrystalline cellulose and calcium carbonate are preferred excipients and their weight ratio directly determines compressibility of tablets. The inventors, by observing actual formulation development process, based on fixed ratio of active ingredients and other auxiliary materials, carefully studied differences in formability of granules/formability of tablets when key auxiliary materials microcrystalline cellulose and calcium carbonate are in different ratios and finally determined a range of ratios of microcrystalline cellulose and calcium carbonate. Please refer to Table 5 for the results.

TABLE 5
Table of formability study of different ratios of
microcrystalline cellulose and calcium carbonate

| microcrystalline | | |
|---|---|---|
| cellulose:calcium | | |
| carbonate | Granule formability | Tablet formability |
| 5:1 | loose particles which can | Tablet formable but |
| | be sifted through mesh | sticking and picking |
| | easily after drying; much | occur very easily; |
| | fine powder of 20%~30% | strict requirement on |
| | of the total amount | environmental |
| | | humidity under 50% |
| 2:1 | loose particles which can | Tablet formable but |
| | be sifted through mesh | sticking and picking |
| | easily after drying; much | occur very easily; |
| | fine powder of 15%~20% | strict requirement on |
| | of the total amount | environmental |
| | | humidity under 50% |
| 1:1 | loose particles which can | Tablet formable |
| | be sifted through mesh | without sticking or |
| | easily after drying; | picking; no |
| | suitable size with less fine | requirement on |
| | powder of 2%~6% of the | environmental |
| | total amount | humidity under 50% |
| 1:2 | Particle agglomerate | Tablet formable |
| | which can be sifted | without sticking or |
| | through mesh after | picking; strict |
| | drying; suitable size with | requirement on |
| | less fine powder of | environmental |
| | 5%~10% of the total | humidity under 50% |
| | amount | |
| 1:5 | Particle agglomerate | Tablet formable with |
| | which can hardly be sifted | sticking or picking; |
| | through mesh after | strict requirement on |
| | drying; much fine powder | environmental |
| | of over 20% of the total | humidity under 50% |
| | amount | |

The results show that tablets are formable when the weight ratio of microcrystalline cellulose and calcium carbonate is between 5:1 and 1:5, but the weight ratio of microcrystalline cellulose and calcium carbonate is preferably 1:1 for the purpose of easier control.

### Example 5: Study of Selection of Auxiliary Material of Granules

Based on physiochemical properties of L-carnitine and characteristics of the dosage form, namely L-carnitine is a flaky crystal and extremely easy to absorb moisture, a sustained release auxiliary material that has moisture absorption resistance shall be adopted. The inventors selected auxiliary materials including, among other, lactose, mannitol, ethanol and citric acid via a great amount of Pharmaceutics Experiments, among which lactose and mannitol are excipients, ethanol is a binding agent, and citric acid is a corrective agent.

Lactose and mannitol are preferred excipients. The inventors, by observing actual formulation development process, based on fixed ratio of active ingredients and other auxiliary materials, carefully studied differences in formability of granules/formability of tablets when key auxiliary materials lactose and mannitol are in different ratios and finally determined a range of ratios of lactose and mannitol. Please refer to Table 6 for the results.

TABLE 6
Table of formability study of different
ratios of lactose and mannitol

| lactose:mannitol | Granule formability |
|---|---|
| 5:1 | particles can easily get agglomerated and cannot be |
| | easily dispersed after drying; difficult to granulate |
| 2:1 | loose particles can easily be dispersed after drying; |
| | easy to granulate; fine powders is 3%~5% of the |
| | total amount |
| 1:1 | loose particles can easily be dispersed after drying; |
| | easy to granulate; fine powders is 5%~10% of the |
| | total amount |
| 1:2 | loose particles can easily be dispersed after drying; |
| | easy to granulate; fine powders is 10%~20% of the |
| | total amount |
| 1:5 | particles can easily get agglomerated and cannot be |
| | easily dispersed after drying; difficult to granulate |

The results show that granules are formable when the weight ratio of lactose and mannitol is between 5:1 and 1:5, but the weight ratio of lactose and mannitol is preferably 2:1 for the purpose of easier preparation.

### Example 6: Study of Selection of Auxiliary Material of Oral Liquid

Based on the characteristics of the dosage form oral liquid and the physiochemical property that L-carnitine has fishlike smell, selected auxiliary materials are mainly corrective agents and sweeteners, including, among others, sodium cyclamate and citric acid. The ratio of sodium cyclamate and citric acid is determined via taste identification by lab personnel. Please refer to table 7 for the results.

TABLE 7
Table of taste study of different ratios
of sodium cyclamate and citric acid

| sodium cyclamate:citric acid | fishlike | sweet | sour |
|---|---|---|---|
| 5:1 | No | Weak | Strong |
| 2:1 | No | Moderate | Strong |
| 1:1 | No | Moderate | Moderate |
| 1:2 | No | Strong | Moderate |
| 1:5 | Yes | Strong | Weak |

The results show that the weight ratio of sodium cyclamate and citric acid between 5:1 and 1:5 provides no irritant taste, but the weight ratio of sodium cyclamate and citric acid is preferably 1:1 for the purpose of best taste.

### Example 7: Compound Preparation L-Carnitine Tablet

Formulation (percentage by weight):

L-carnitine: 16%

trimetazidine hydrochloride: 10%

microcrystalline cellulose: 50%

calcium carbonate: 10%

cross-linked polyvinylpyrrolidone: 4%

polyvinylpyrrolidone: 5%

talcum powder: 4%

magnesium stearate: 1%

Process:
1) Let L-carnitine, trimetazidine hydrochloride, microcrystalline cellulose, calcium carbonate, cross-linked polyvinylpyrrolidone, polyvinylpyrrolidone and talcum powder be sifted through 100 mesh sieve, respectively, for further use;
2) Weighing according to the formulation L-carnitine, trimetazidine hydrochloride, microcrystalline cellulose, calcium carbonate and polyvinylpyrrolidone, which are then evenly mixed;
3) Adding an appropriate amount of 80% ethanol solution, preparing soft material and have it sifted through 20 mesh sieve to prepare granules;
4) Placing wet granules in a forced air drier at 50° C. and have them dried for four hours;
5) After drying is complete, the granules are taken out and sifted through 20 mesh sieve for granulation. Then adding prescribed amount of magnesium stearate, talcum powder and cross-linked polyvinylpyrrolidone, which are then mixed evenly;
6) Selecting appropriate punch according to requirement on tablet weight and tableting.

### Example 8: Compound Preparation L-Carnitine Tablet

Formulation (percentage by weight):

L-carnitine: 80%

trimetazidine hydrochloride: 0.1%

microcrystalline cellulose: 6%

calcium carbonate: 3%

cross-linked polyvinylpyrrolidone: 2%

polyvinylpyrrolidone: 4%

talcum powder: 4%

magnesium stearate: 0.9%

Process:

The same as that in example 7

### Example 9: Compound Preparation L-Carnitine Tablet

Formulation (percentage by weight):

L-carnitine: 65%

trimetazidine hydrochloride: 10%

microcrystalline cellulose: 5%

calcium carbonate: 10%

cross-linked polyvinylpyrrolidone: 2%

sodium carboxymethyl cellulose: 2%

talcum powder: 5%

magnesium stearate: 1%

Process:
1) Let L-carnitine, trimetazidine hydrochloride, microcrystalline cellulose, calcium carbonate, cross-linked polyvinylpyrrolidone, sodium carboxymethyl cellulose and talcum powder be sifted through 100 mesh sieve, respectively, for further use;
2) Weighing according to the formulation L-carnitine, trimetazidine hydrochloride, microcrystalline cellulose, calcium carbonate and sodium carboxymethyl cellulose, which are then evenly mixed;
3) Adding an appropriate amount of 70% ethanol solution, preparing soft material and have it sifted through 20 mesh sieve to prepare granules;
4) Placing wet granules in a forced air drier at 50° C. and have them dried for four hours;
5) After drying is complete, the granules are taken out and sifted through 20 mesh sieve for granulation. Then adding prescribed amount of magnesium stearate, talcum powder and cross-linked polyvinylpyrrolidone, which are then mixed evenly;
6) Selecting appropriate punch according to requirement on tablet weight and tableting.

### Example 10: Compound Preparation L-Carnitine Tablet

Formulation (percentage by weight):

L-carnitine: 65%

trimetazidine hydrochloride: 0.2%

microcrystalline cellulose: 4%

calcium carbonate: 20%

cross-linked sodium carboxymethyl cellulose: 2%

sodium carboxymethyl cellulose: 2%

talcum powder: 5%

magnesium stearate: 0.8%

Process:

The same as that in example 9

### Example 11: Compound Preparation L-Carnitine Tablet

Formulation (percentage by weight):

L-carnitine: 75%

trimetazidine hydrochloride: 0.75%

microcrystalline cellulose: 8%

calcium carbonate: 8%

cross-linked sodium carboxymethyl cellulose: 2.25%

sodium carboxymethyl cellulose: 2%

talcum powder: 3%

magnesium stearate: 1%

Process:

The same as that in example 9

### Example 12: Compound Preparation L-Carnitine Tablet

The formulation and process are the same as those in example 9, except that L-carnitine in example 11 is substituted by acetyl-L-carnitine or propionyl-L-carnitine.

### Example 13: Compound Preparation L-Carnitine Tablet

The formulation and process are the same as those in example 9, except that L-carnitine in example 11 is substituted by a salt formed by L-carnitine and one of the following: hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid or p-toluene sulfonic acid.

### Example 14: Compound Preparation L-Carnitine Tablet

The formulation and process are the same as those in example 9, except that trimetazidine hydrochloride in example 11 is substituted by a salt formed by trimetazidine hydrochloride and one of the following: hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid or p-toluene sulfonic acid.

### Example 15: Combined Package Formulation

Separately prepare or purchase L-carnitine and trimetazidine hydrochloride formulations, as shown in Table 8.

TABLE 8
L-carnitine and trimetazidine hydrochloride
formulations in different specifications

| L-carnitine tablet | trimetazidine hydrochloride tablet |
|---|---|
| L-carnitine tablet 0.25 g | trimetazidine hydrochloride tablet 2 mg |
| L-carnitine tablet 0.333 g | trimetazidine hydrochloride tablet 3 mg |
| L-carnitine tablet 0.5 g | trimetazidine hydrochloride tablet 5 mg |
| L-carnitine tablet 1 g | trimetazidine hydrochloride tablet 10 mg |
| L-carnitine tablet 2 g | trimetazidine hydrochloride tablet 15 mg |
| | trimetazidine hydrochloride tablet 20 mg |
| | trimetazidine hydrochloride tablet 30 mg |

### Example 16: Compound Preparation L-Carnitine Granule

Formulation (percentage by weight):

L-carnitine: 8%

trimetazidine hydrochloride: 1%

lactose: 50%

mannitol: 10%

dextrin: 21%

citric acid: 3%

sodium cyclamate: 2%

polyvinylpyrrolidone: 5%

Process:
1) Let L-carnitine, trimetazidine hydrochloride, lactose, mannitol, polyvinylpyrrolidone, dextrin, citric acid and sodium cyclamate be sifted through 100 mesh sieve, respectively, for further use;
2) Weighing according to the formulation L-carnitine, trimetazidine hydrochloride, lactose, mannitol, polyvinylpyrrolidone, dextrin, citric acid and sodium cyclamate, which are then evenly mixed;
3) Adding an appropriate amount of 70% ethanol solution, preparing soft material and have it sifted through 20 mesh sieve to prepare granules;
4) Placing wet granules in a forced air drier at 50° C. and have them dried for four hours;
5) After drying is complete, the granules are taken out and sifted through 20 mesh sieve for granulation.

### Example 17: Compound Preparation L-Carnitine Granule

Formulation (percentage by weight):

L-carnitine: 50%

trimetazidine hydrochloride: 0.1%

lactose: 20%

mannitol: 10%

dextrin: 11%

citric acid: 3%

sodium cyclamate: 0.9%

polyvinylpyrrolidone: 5%

Process:

The same as that in example 16

### Example 18: Compound Preparation L-Carnitine Granule

Formulation (percentage by weight):

L-carnitine: 16%

trimetazidine hydrochloride: 0.25%

lactose: 20%

mannitol: 40%

dextrin: 25%

citric acid: 3%

sodium cyclamate: 1%

banana essence: 0.75%

polyvinylpyrrolidone: 4%

Process:

The same as that in example 16

### Example 19: Compound Preparation L-Carnitine Granule

Formulation (percentage by weight):

L-carnitine: 20%

trimetazidine hydrochloride: 0.2%

lactose: 40%

mannitol: 20%

dextrin: 15%

citric acid: 1%

sodium cyclamate: 1%

banana essence: 0.8%

polyvinylpyrrolidone: 2%

Process:

The same as that in example 16

### Example 20: Compound Preparation L-Carnitine Granule

The formulation and process are the same as those in example 16, except that L-carnitine in example 19 is substituted by a salt formed by L-carnitine and one of the following: hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid or p-toluene sulfonic acid.

### Example 21: Compound Preparation L-Carnitine Granule

The formulation and process are the same as those in example 16, except that trimetazidine hydrochloride in example 19 is substituted by a salt formed by trimetazidine hydrochloride and one of the following: hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid or p-toluene sulfonic acid.

### Example 22: Combined Package Formulation

Separately prepare or purchase L-carnitine and trimetazidine hydrochloride formulations, as shown in Table 9.

TABLE 9
L-carnitine and trimetazidine hydrochloride
formulations in different specifications

| L-carnitine granule | trimetazidine hydrochloride granule |
|---|---|
| L-carnitine granule 0.25 g | trimetazidine hydrochloride granule 2 mg |
| L-carnitine granule 0.333 g | trimetazidine hydrochloride granule 3 mg |
| L-carnitine granule 0.5 g | trimetazidine hydrochloride granule 5 mg |
| L-carnitine granule 1 g | trimetazidine hydrochloride granule 10 mg |
| L-carnitine granule 2 g | trimetazidine hydrochloride granule 15 mg |
| | trimetazidine hydrochloride granule 20 mg |
| | trimetazidine hydrochloride granule 30 mg |

### Example 23: Compound Preparation L-Carnitine Oral Liquid

Formulation (percentage by weight/volume):

L-carnitine: 5%

trimetazidine hydrochloride: 0.6%

lactose: 10%

mannitol: 10%

citric acid: 5%

sodium cyclamate: 1%

potassium sorbate: 0.02%

distilled water: appropriate

Process:

Weighing the raw auxiliary material according to the formulation. After being dissolved in an appropriate amount of distilled water, adding more distilled water to dilute it to a predetermined solubility.

### Example 24: Compound Preparation L-Carnitine Oral Liquid

Formulation (percentage by weight/volume):

L-carnitine: 60%

trimetazidine hydrochloride: 0.1%

lactose: 10%

mannitol: 10%

citric acid: 4%

sodium cyclamate: 2%

potassium sorbate: 0.02%

distilled water: appropriate

Process:

Weighing the raw auxiliary material according to the formulation. After being dissolved in an appropriate amount of distilled water, adding more distilled water to dilute it to a predetermined solubility.

### Example 25: Compound Preparation L-Carnitine Oral Liquid

Formulation (percentage by weight/volume):

L-carnitine: 30%

trimetazidine hydrochloride: 0.3%

lactose: 10%

mannitol: 10%

citric acid: 4%

sodium cyclamate: 2%

potassium sorbate: 0.02%

distilled water: appropriate

Process:

Weighing the raw auxiliary material according to the formulation. After being dissolved in an appropriate amount of distilled water, adding more distilled water to dilute it to a predetermined solubility.

### Example 26: Compound Preparation L-Carnitine Oral Liquid

Formulation (percentage by weight/volume):

L-carnitine: 10%

trimetazidine hydrochloride: 0.1%

lactose: 5%

mannitol: 15%

citric acid: 2%

sodium cyclamate: 2%

potassium sorbate: 0.02%

distilled water: appropriate

Process:

Weighing the raw auxiliary material according to the formulation. After being dissolved in an appropriate amount of distilled water, adding more distilled water to dilute it to a predetermined solubility.

### Example 27: Compound Preparation L-Carnitine Oral Liquid

The formulation and process are the same as those in example 26, except that L-carnitine in example 26 is substituted by a salt formed by L-carnitine and one of the following: hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid or p-toluene sulfonic acid.

### Example 28: Compound Preparation L-Carnitine Oral Liquid

The formulation and process are the same as those in example 26, except that trimetazidine hydrochloride in example 26 is substituted by a salt formed by trimetazidine hydrochloride and one of the following: hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid or p-toluene sulfonic acid.

### Example 29: Combined Package Formulation

Separately prepare or purchase L-carnitine and trimetazidine hydrochloride formulations, as shown in Table 10.

TABLE 10
L-carnitine and trimetazidine hydrochloride
formulations in different specifications

| L-carnitine oral liquid | trimetazidine hydrochloride oral liquid |
|---|---|
| L-carnitine oral liquid | trimetazidine hydrochloride oral liquid 2 mg |
| 0.25 g | |
| L-carnitine oral liquid | trimetazidine hydrochloride oral liquid 3 mg |
| 0.333 g | |
| L-carnitine oral liquid | trimetazidine hydrochloride oral liquid 5 mg |
| 0.5 g | |
| L-carnitine oral liquid | trimetazidine hydrochloride oral liquid 10 mg |
| 1 g | |
| L-carnitine oral liquid | trimetazidine hydrochloride oral liquid 15 mg |
| 2 g | |
| | trimetazidine hydrochloride oral liquid 20 mg |
| | trimetazidine hydrochloride oral liquid 30 mg |

What is claimed is:

1. A method for increasing hypoxia tolerance in a subject suffering from hypoxia, comprising:
creating a pharmaceutical composition which comprises (a) a first active ingredient selected from the group consisting of L-carnitine and derivatives and pharmaceutically acceptable salts thereof, (b) a second active ingredient selected from the group consisting of trimetazidine and derivatives and pharmaceutically acceptable salts thereof, and (c) a pharmaceutically acceptable auxiliary material, and wherein the weight ratio of the first active ingredient to the second active ingredient is within the range of 50-4000:1; and
administering the pharmaceutical composition to the subject.

2. The method of claim 1, wherein the first active ingredient is selected from the group consisting of L-carnitine, acetyl-L-carnitine, propionyl-L-carnitine and pharmaceutically acceptable salts thereof.

3. The method of claim 1, wherein the pharmaceutically acceptable salts of trimetazidine, L-carnitine or derivatives thereof are selected from the group consisting of their salts formed with hydrochloric acid, hydrobromic acid, hydroiodic acid, sulfuric acid, nitric acid, phosphoric acid, acetic acid, maleic acid, fumaric acid, citric acid, oxalic acid, succinic acid, tartaric acid, malic acid, mandelic acid, trifluoroacetic acid, pantothenic acid, methane sulfonic acid and p-toluene sulfonic acid.

4. The method of claim 1, wherein the pharmaceutical composition is orally administered to the subject in a form selected from the group consisting of tablets, granules and liquids.

5. The method of claim 4, wherein the pharmaceutical composition is administered to the subject in the form of a tablet.

6. The method of claim 4, wherein the pharmaceutical composition is orally administered to the subject in the form of a granule.

7. The method of claim 4, wherein the pharmaceutical composition is orally administered to the subject in the form of a liquid.

8. The method of claim 1, wherein the oral pharmaceutical composition is in a combined package.

9. The method of claim 1, wherein the ratio of the first active ingredient to the second active ingredient is within the range of 66-4000:1.

10. The method of claim 1, wherein the ratio of the first active ingredient to the second active ingredient is within the range of 66-100:1.

11. The method of claim 1, wherein the ratio of the first active ingredient to the second active ingredient is 100:1.

12. The method of claim 1, wherein the pharmaceutical composition increases blood oxygen saturation in the subject, and wherein the ratio of the first active ingredient to the second active ingredient is within the range of 50-300:1.

13. The method of claim 12, wherein the ratio of L-carnitine or derivative thereof or pharmaceutically acceptable salt thereof and trimetazidine or pharmaceutically acceptable salt thereof is 100:1.

14. The method of claim 9, wherein the subject is an adult, and wherein the pharmaceutical composition is administered to the subject in a daily dosage of 10-500 mg/kg for the first active ingredient, and 0.1-1 mg/kg for the second active ingredient.
